(19) United States
(12) Patent Application Publication
(10) Pub. No.: US 2005/0187429 A1
(43) Pub. Date: Aug. 25, 2005

(54) Vaginal rejuvenation

(76) Inventors: Dix P. Poppas, Larchmont, NY (US); Michael T. Milbocker, Holliston, MA (US)

Correspondence Address: Promethean Surgical Devices LLC, Suite G, 3 Gill St., Woburn, MA, 01801-1720, US

(21) Appl. No.: 11/062,779

(22) Filed: Feb. 22, 2005

Related U.S. Application Data
(60) Provisional application No. 60/546,787, filed on Feb. 23, 2004.

(51) Int. Cl. A61F 2/02

(52) U.S. Cl. 600/30

(57) ABSTRACT

A tissue reinforcement therapy to make the vaginal vault smaller and possibly increase and/or restore sexual gratification is described. The therapy involves augmenting the distal third of the vagina, the orgasmic platform, the external vaginal opening and the perineal area with an injectable in situ polymerizing liquid.

This application claims the benefit of the priority of co-pending provisional U.S. application 60/546,787, filed Feb. 23, 2004, which is hereby incorporated by reference.

### FIELD OF THE INVENTION

This invention relates to delivery of a class of medical liquids into the body, and which solidify inside the body to yield a solid or gel. The invention also relates to the method of using such liquids in medical applications such as filling of various locations in the pelvis to improve the performance of the tissue in a bodily function. In particular, a method and device are described for enhancing the tension in vaginal tissues and pelvic floor.

### BACKGROUND OF THE INVENTION

Vaginal tissues and the pelvic floor can be weakened and made loose because of multiple child births, aging, or genetic disorders. Generally the inner and outer dimensions of the vagina increase with age. The vagina generally becomes more flaccid during these processes, which can interfere with sexual responses. A current therapy for this condition is posterior-vaginal repair. The posterior structures are dissected out and excess vaginal tissue is excised, and the pelvic floor is reinforced by approximating the muscle layers. This is a painful operation with a prolonged recovery.

In U.S. Pat. No. 6,165,108, the use of a dumbbell-shaped device for exercising the musculature of the vagina is proposed. Several cited references described variants on these themes. No evidence of actual increase in tone is presented. U.S. Pat. No. 6,469,016 describes the use of prostaglandins and related chemicals to temporarily affect local lubricity, and also affect local muscle tone. The first effect is plausible. In neither of these cases is there a long-lasting effect of the therapy. Zunker (U.S. Pat. No. 6,770,025) describes a device for insertion into the vagina to improve urinary continence. Compositions for bulking tissue to treat incontinence or GERD (gastro-esophageal reflux disease) are described by Durgin (U.S. Pat. No. 6,591,838), Goupil et al (U.S. Pat. No. 6,652,883), Vogel et al (U.S. Pat. No. 6,660,301, and by applicants (U.S. Pat. No. 6,296,607). However, there currently is apparently no description of a permanent therapy for returning a vagina to pre-childbirth tension without surgical removal of tissue. It would be desirable to be able to remove vaginal flaccidity on a long-term basis without requiring surgery.

### SUMMARY OF THE INVENTION

The present invention relates to surgical adhesives, methods of using surgical adhesives, kits comprising surgical adhesives, and methods of using surgical adhesives to treat vaginal looseness.

One aspect of the invention involves a surgical adhesive used as a tissue filling or augmentation medium to decrease the internal diameter of the vagina. The surgical adhesive cures in place in the body to form an elastic, malleable solid capable of displacing tissue and thereby increasing the tone of the vagina.

The surgical adhesive has several embodiments. In one embodiment, the surgical adhesive may be an isocyanate-capped polyol. In another embodiment, the surgical adhesive may be absorbable. In another embodiment, the surgical adhesive may contain therapeutic substances such as antibiotics and substances that promote healing or reduce pain. In another embodiment, the adhesive may contain bacitracin. In another embodiment the adhesive contains neomycin. In still another embodiment the adhesive may contain an anesthetic agent such as Marcaine.

In some embodiments, the adhesive is 2-part and is mixed at time of use. In some of these embodiments, one part may be absorbable and the other part non-absorbable and the two parts immiscible such that a mixture of the two parts consists in regions substantially comprising only one part. In this embodiment, both parts can separately polymerize within the body. However, in their mixed state they form a layer or depot comprising both regions of absorbable polymer and regions of non-absorbable polymer. Over time the absorption of the absorbable fraction results in a plurality of openings in the polymerized layer, which mimics a mesh structure and enhances vascularization and tissue ingrowth.

In some embodiments the in situ polymerizing fluid does not chemically bond to tissue and thus is localized entirely by mechanical bonding to tissue. Mechanical bonding occurs when the liquid prepolymer infiltrates small-scale structures within the tissue and then solidifies. In other embodiments, the in situ polymerizing fluid chemically bonds to tissue. In another embodiment of the invention the surgical adhesive contains a strengthening agent such as flock or solid particulate.

In another aspect, the invention relates to a kit for performing vaginal rejuvenation. The kit comprises a sterile in situ polymerizing fluid or surgical adhesive, a delivery device, and one or more vaginal shape stents. A vaginal shape stent is a removable object placed in the vagina and having a shape which places the vagina in a shape which the implantation of adhesive is intended to maintain.

Yet another aspect of the invention involves a method of using a surgical adhesive for vaginal rejuvenation. The method includes depositing surgical adhesive between various tissue layers of the female pelvis to fix tissue layers, to displace the inner wall of the vagina, and to fill form an elastic shape-inducing implant within these layers.

In another aspect of the invention, reduction of the vaginal inner diameter is combined with a method of stabilizing the pelvic floor. In this method a continuous layer of adhesive is applied with one part of the layer in contact with the tissue to be reinforced and another part in contact with a supporting structure. Typical supporting structures include fascia, ligament, bone, and muscle. The supporting structure must be located so that when the surgical adhesive cures in the therapeutic position, forces generated by these supporting structures are sufficient to maintain the therapeutic position of the tissue. In some embodiments the surgical adhesive bonds to the arcus tendinous fascia pelvis. In other embodiments, the adhesive bonds to the ileal pectineal muscle group. In still other embodiments, the adhesive bonds to the pubococcygeous muscles.

The foregoing and other objects, aspects, features, and advantages of the invention will become more apparent from the following description and from the claims.

### BRIEF DESCRIPTION OF THE FIGURES

FIG. 1 is a schematic drawing of the vagina and surrounding tissues.

FIG. 2 shows preferred locations of depots of resilient polymeric material and a syringe for injecting the prepolymer.

### DETAILED DESCRIPTION OF THE INVENTION

The present invention relates to surgical adhesives for use in modification of the vaginal wall and vaginal introitus. More particularly, the surgical adhesive is useful in modifying the shape, dimension or tone of the vagina. The surgical adhesive of the present invention may be any known in the art. One class of adhesives comprises natural proteins, and proteins with synthetic groups or crosslinkers (such as glutaraldehyde), used as sealants or adhesives, including without limitation collagen, fibrin, albumin, elastin, and derivatives of these or other proteins. Another class of adhesives comprises cyanoacrylates or other in-situ polymerizable acrylic compositions. Another class of adhesives comprises polymers, particularly polyols functionalized with succinimidyl groups. Another, preferred, class of adhesives comprises polyols and polyester polyols derivatized with polyisocyanates to form self-curing adhesives and bulking agents with tissue-bonding capability, as described, for example, in U.S. Pat. No. 6,296,607, U.S. Pat. No. 6,254,327, U.S. 2002/0049363, U.S. 2003/0135238 and U.S. 2003/0188755, which are incorporated herein by reference.

Polymerization times can be adjusted by selection of various components of the polymerizing material. In a preferred embodiment, the material consists of a polyisocyanate capped polyol and additional free polyisocyanate, essentially as described in the above references. The free isocyanate is a low molecular weight polyisocyanate, typically a compound such as toluene diisocyanate, but optionally a larger molecule, such as the reaction product of a commercial diisocyanate with a trifunctional or tetrafunctional low molecular weight material such as glycerol, trimethylol propane, erythritol, etc. Preferably the molecular weight of the free isocyanate material is below about 1000 daltons, more preferably below about 500 daltons. Alternatively, the molecular weight of the free isocyanate component is less than about 20%, preferably less than about 10%, more preferably less than about 5%, of the number-average molecular weight of the polymeric isocyanate component.

The capped polyol is multifunctional, and typically trifunctional. The polyol may be any of various biocompatible substances, including particularly polymers of ethylene oxide, propylene oxide, ethylene glycol, and copolymers of these. The polyisocyanate is typically difunctional. Fast reacting formulations use an aromatic diisocyanate such as toluene diisocyanate. Slow reacting formulations use an aliphatic diisocyanate such as isophorone diisocyanate. Alternatively, the polymerization time can be adjusted by selection appropriate molecular weight polyols. The higher molecular weight polyols produce lower viscosity capped reaction products and faster reacting solutions.

These polymer mixtures typically and preferably have the added advantage of being water-soluble. Their water solubility enables them to be injected into tissue to polymerize with the tissue; or alternatively to solidify as gels to stabilize tissue. The material acts as a self-sealing fluid when injected into cavities within the body.

The surgical adhesive may be incorporated with or delivered in addition to electrolytic solutions (such as saline), contrast media (such as Conray meglumine iothalamate, or tantalum powder), pharmaceutical agents for therapy or prophylaxis, disinfectants and other antimicrobial agents, and/or filling or binding materials.

The surgical adhesive may be mixed with flock fibers to increase its tear resistance after polymerization without increasing prohibitively the injectability of the solution, for example by increase in its viscosity. Additionally, selecting solutions with a higher ratio of polymerizing agent to aqueous solution creates a more rigid gel. Typically, the gel modulus can be controlled from a very loose gel to one of 50D Shore or greater by control of concentration and composition.

The surgical adhesives of the present invention may be made absorbable or biodegradable by incorporating in the polyol backbone any of the known monomeric groups that form polymers that contain bonds which are biodegradable, i.e., which gradually break under the natural conditions found in the body, so that the polymers eventually decrease in molecular weight sufficiently to be absorbed or excreted. Such materials are also known as absorbable or as bioabsorbable. Biodegradable groups include without limitation polyesters formed from hydroxycarboxylic acids, particularly aliphatic monohydroxymonocarboxylic acids with hydroxyl substitution at the alpha, beta and epsilon carbons, and most especially lactic acid, glycolic acid, and hydroxycaproic acid, particularly as the lactone or cyclic dimer forms. Other classes of biodegradable bonds or polymers include polyanhydrides formed from dicarboxylic aliphatic and aromatic acids; polycarbonates from aliphatic cyclic carbonates, such as trimethylene carbonate; polypeptides and proteins; and polymers containing other labile groups in the backbone, such as poly phosphoesters. Preferred degradable polymer compositions include polyglycolic acid, polylactic acid, polycaprolactone, poly-alpha amino acids including proteins and polypeptides, and blends and copolymers thereof. Incorporation of degradable groups into polymeric adhesives is described in our co-pending application U.S. 2004/0068078.

Because the removal or amelioration of vaginal flaccidity by the use of adhesives is generally meant to be permanent, preferred adhesives will be non-biodegradable. However, inclusion of degradable domains within an initially formed layer of polymer can promote permanent tissue-based healing by creating spaces allowing ingrowth of tissue. One method of providing such domains is to include a biodegradable particulate material in the surgical adhesive. Another way is to make a crosslinkable biodegradable polymer that is phase-incompatible with the non-degradable adhesive component, for example by being water-insoluble or by being based on a phase-incompatible backbone, for example a dextran-based biodegradable adhesive mixed with a polyalkylene glycol-based non-degradable adhesive component.

### EXAMPLE 1

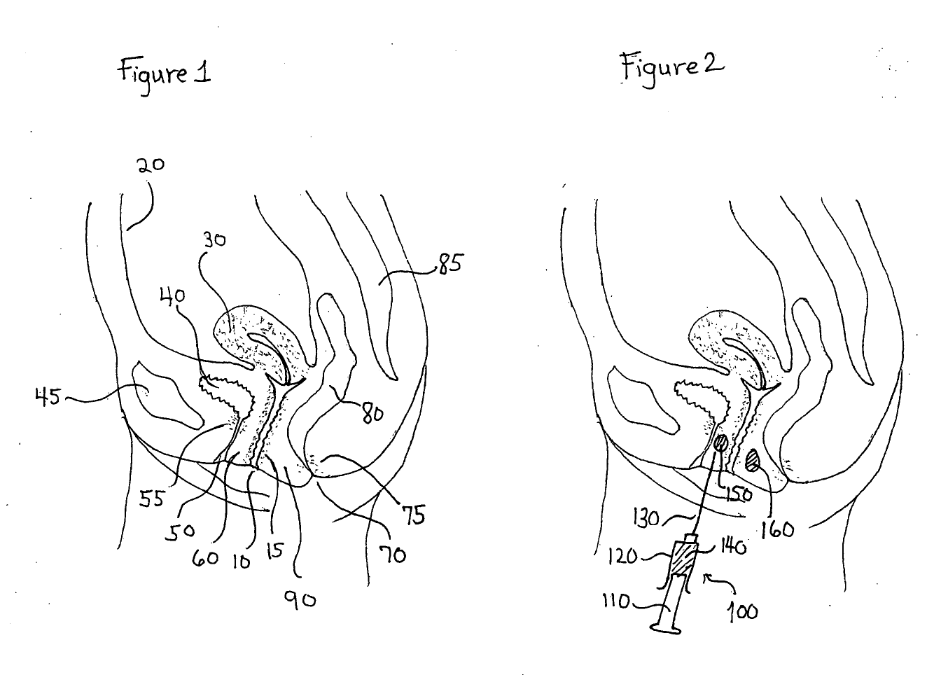

A procedure for vaginal rejuvenation is described, which is expected to be effective when tested. FIG. 1 schematically shows the vagina 10 and the surrounding tissues, including the muscles 15 surrounding the vagina, the peritoneum 20 and the uterus 30; the bladder 40, the pubic bone 45, the urethra 50, and the urethral sphincter 55; the tissues 60 between the urethra 50 and the vagina 10; the anus 70, the anal sphincter 75 and the rectum 80; the spine 85; and the tissues 90 between the vagina and the rectum.

In FIG. 2, a syringe 100 is shown with a plunger 110, a barrel 120, a hub 130, and carrying a needle 150. The syringe is loaded with an implant-forming material 140. A deposit 150 of injected implant material is shown, and a previously-formed deposit is shown at 160.

To modify the vestibule of the vagina, a syringe 100 with a needle 150 that is, for example, _inch (1.2 cm) long or longer, and 23 G or larger in diameter, is loaded with an implant-forming polymer. The insertion of the needle is guided by a cystoscope or by eye. In one approach, the point of the needle is placed in the vaginal wall and advanced into the interdigitating fibers of the perineum. Encircling the vagina and urethra is the urethrovaginal sphincter. In some preferred implant locations, labeled as 150 in FIG. 2, the needle is advanced between the urethrovaginal sphincter and the muscular wall of the vagina. Surgical adhesive, which forms an implant after curing, is delivered to this site. The elastic but incompressible nature of the surgical adhesive displaces the wall of the vagina towards its axis while in many cases displacing the urethrovaginal sphincter away from its axis. Depending on the extent of adhesive delivery and the degree of mobilization of the urethrovaginal sphincter, the procedure may also serve to place tension on the urethra. This may also be beneficial in the control of urinary incontinence, which like vaginal looseness is associated with childbirth. Injection in the tissue 90 between the rectum and the vagina can also be useful, depending on the degree of flaccidity to be corrected.

The vagina may also be displaced laterally by injecting adhesive between the muscular wall of the vagina and the puborectalis and pubococcugeus muscles or into the soft tissue immediately surrounding the vagina. The soft tissue of the vaginal introitus may also be injected anywhere from the rectovaginal septum to the periurethral area. The vagina may be shortened by injecting adhesive into the wall of the anterior part of the vaginal fornix.

In any of these procedures, introducing vaginal forming devices of a preferred shape, which may be known as vaginal stents, may control the internal shape of the vagina. When the stent is present during application of surgical adhesive, the resulting layer of cured adhesive formed in the muscle and/or soft tissue layers of the vagina and/or adjacent tissue, which is elastic and malleable, will have a shape conforming to that of the stent, and will tend to resist distortion away from the stent-induced preferred shape.

Although this invention has been described in terms of certain preferred embodiments, other embodiments will be apparent to those of ordinary skill in the art and are considered within the scope of this invention.

1. A surgical method of decreasing flaccidity in the vagina, the method comprising the steps of:
a) selecting at least one site in tissue for treatment, the site being selected from one or more of the tissues of the vagina and the tissues surrounding the vagina;
b) introducing an applicator tip of a delivery device into said site or sites;
c) introducing a polymeric surgical adhesive into said tissue site or sites from said delivery device via said applicator tip,
wherein said adhesive cures to form an implant at the site where it is injected.

2. The method of claim 1 wherein the adhesive is introduced between the urethrovaginal sphincter and the muscular wall of the vagina.

3. The method of claim 1 wherein sufficient adhesive is delivered to tighten both the vagina and the urethra.

4. The method of claim 1 wherein the delivery device is a syringe, and the applicator tip is a syringe needle.

5. The method of claim 1 wherein at least one component of the surgical adhesive is biodegradable.

6. The method of claim 1 wherein the surgical adhesive is based on a polymer comprising reactive isocyanate groups.

7. The method of claim 6 wherein the surgical adhesive further comprises low molecular weight polyisocyanate.

8. The method of claim 1 wherein the polymeric polyol comprises a polyether polyol.

9. The method of claim 8 wherein the polymeric polyol comprises a polymer or copolymer of one or more alkylene oxides.

10. The method of claim 9 where at least about 70% by weight of the polymeric component of the surgical adhesive is derived from ethylene oxide monomers.

11. The method of claim 1 wherein the surgical adhesive comprises reactive groups that react with two characteristic speeds, wherein a first fast reacting component reacts with tissue and a second slower reacting component reacts with other adhesive components to crosslink the adhesive.

12. The method of claim 1 wherein the adhesive further contains reinforcing materials.

13. The method of claim 1 further comprising using a vaginal stent forming device to shape the vagina during its reinforcement.

14. A surgical repair kit for vaginal rejuvenation comprised of one or more surgical adhesives, one or more delivery devices, and one or more applicator tips, supplied in a container which also contains, or bears on its surface, or both, written material describing how to use the kit to rejuvenate the vagina.

15. The kit of claim 14 wherein the delivery device is a syringe.

16. The kit of claim 14 wherein the applicators tip is a hypodermic needle.

17. The kit of claim 14 wherein two surgical adhesives are included, one of which is immiscible with the second, and the kit further comprising a mixing means that will temporarily create a suspended mixed state that can then be loaded into the delivery means.

18. The kit of claim 14 wherein the surgical adhesive comprises an isocyanate capped polyol.

19. The kit of claim 18 wherein a portion of the polyol is bioabsorbable.

20. The kit of claim 14 further comprising a vaginal stent.

21. The kit of claim 14 further comprising a reinforcing material for the adhesive.

22. The use of a vaginal stent device for controlling the final shape of a vagina during an operation, the device being a solid material formed into a shape to which the vagina is to be conformed during said operation.
